(19) United States
(12) Patent Application Publication
(10) Pub. No.: US 2002/0031812 A1
(43) Pub. Date: Mar. 14, 2002

(54) Process for production of biopolymer

(75) Inventors: Richard Lapointe, Montreal (CA); Alex Lambert, Montreal (CA); Louise Savard, Montreal (CA)

Correspondence Address: OGILVY RENAULT, 1981 MCGILL COLLEGE AVENUE, SUITE 1600, MONTREAL, QC, H3A2Y3, CA

(73) Assignee: La Societe Novartem inc., Chicoutimi (CA)

(21) Appl. No.: 09/949,881

(22) Filed: Sep. 12, 2001

Related U.S. Application Data
(60) Provisional application No. 60/230,918, filed on Sep. 13, 2000.

(51) Int. Cl. C12P 7/62; A61K 31/19

(52) U.S. Cl. 435/135; 514/557

(57) ABSTRACT

The present invention relates to a process of production of polyhydroxyalkanoate (PHA) by incubating PHA producing microorganisms in a medium containing starch, starch extracts, or derivatives as sources of carbon. The process comprises also the synthesis of derived compounds belonging to the chemical family of PHA.

### BACKGROUND OF THE INVENTION

(a) Field of the Invention

The invention relates to polymer production and in particular to a process for microbiologically producing poly-3-hydroxyalkanoate (PHAs) and derivatives thereof.

(b) Description of Prior Art

There has been considerable interest in recent years in the use of biodegradable polymers to address concerns over plastic waste accumulation. The potential worldwide market for biodegradable polymers is enormous. Some of the markets and applications most amenable to the use of such biopolymers involve those having single, short use applications, including packaging, personal hygiene, garbage bags, and others. These applications are ideally suited for biodegradation through composting.

Also, polymers find uses in a variety of plastic articles including films, sheets, fibers, foams, molded articles, adhesives and many other specialty products. For applications in the areas of packaging, agriculture, household goods and personal care products, polymers usually have a short (less than 12 months) use cycle. For example, in food packaging, polymers play the role of a protective agent and are quickly disposed of after the contents are consumed. Hygiene products like sanitary or diapers are immediately discarded once the product is used.

The majority of this plastic material ends up in the solid waste stream, headed for rapidly vanishing and increasingly expensive landfill space. While some efforts at recycling have been made, the nature of polymers and the way they are produced and convened to products limits the number of possible recycling applications. Repeated processing of even pure polymer results in degradation of material and consequently poor mechanical properties. Different grades of chemically similar plastics (e.g., polyethylene of different molecular weights, as used in milk jugs and grocery bags) mixed upon collection can cause processing problems that make the reclaimed material inferior or unusable.

Polyhydroxyalkanoates (PHAs) and more specifically poly-3-hydroxybutyrate (P3HB), a short side chain length polymer, have been known for years as being naturally synthesized biodegradable, biocompatible thermoplastics. These are bacterial polyesters used as energy storage when microorganisms are submitted to adverse growth conditions. The polymers are then formed as intracellular granules that can accumulate to 80 percent of the cell mass. The various monomers formulae are commonly reduced to:

—OCHR(CH₂)ₙ—CO—

wherein n is an integer ranging from 1 to 5 and R consists either of a hydrogen or an alkyl group. The physical properties of P3HB (and mostly the copolymer P3Hn-co-3HV) have shown to compare those of polypropylene (PP) such that conventional processing techniques like melting, extrusion and blow forming may be used. Other polymers known as medium side chain length (mcl) behave like elastomers and therefore aim at different applications.

So far, PHAs have been produced through fermentation processes followed by extraction and purification methods. Although research is undergoing toward production in transgenic plants, it is expected that robustness and versatility of bioprocesses will claim to make fermentation the preferred technique for potential medium to large-scale production.

Until recently the limitations to viable commercial production of these bioplastics were mainly due to production costs as compared to synthetic petroleum based polymers. At present, it becomes well recognized that the properties of the PHAs are sought for specific applications and high value-added products in the fields of specialty packaging, cosmetics and biomedicals. Nevertheless, the production costs are still considered to be a major constraint to the development of a profitable industry. In order to address this drawback, it is necessary to make use of cheap carbon sources that are also abundant.

It would be highly desirable to be provided with method for producing a biologically degradable and biocompatible polyhydroxyalkanoate and derivatives thereof.

### SUMMARY OF THE INVENTION

One aim of the present invention is to provide a process for production of polyhydroxyalkanoate (PHA) which comprises the step of incubating a PHA-producing microorganism in a medium comprising crude, isolated or treated starch and recovering PHA from the microorganism.

In accordance to the present invention, is provided a biomass containing starch which is processed to render the starch available sufficiently in a soluble form and/or in the form of an extract to be chemically biochemically, enzymatically and/or biologically treated.

In accordance with the present invention there is provided the starch is further hydrolyzed before incubation of PHA producing microorganisms.

Another aim of the present invention is to provide a process for producing polyhydroxyalkanoate selected from the group consisting of polymer of hydroxyalkanoic acid, hydroxybutyric acid, hydroxyvaleric acid, and copolymers thereof, wherein the copolymers may be poly(hydroxybutyrate-co-hydroxyvalerate) (PHBHV) poly(3-hydroxybutyrate-co-4-hydroxybutyrate) (P3HB4HB), polymers and/or copolymers of hydroxyterminated polyhydroxybutyrate (PHB-OH), heteropolmers thereof, and any other polymers having a chemical structure consistent with the general formula previously described.

In accordance with the present invention another object is to provide a A polyhydroxyalkanoate (PHA) produced by incubation of at least one strain of PHA-producing microorganism in a culture medium comprising starch and/or a derivative thereof.

The biomass of the present invention may be selected from the group consisting of plants, wastewater, washed waters, potatoes, and by-products or derivatives thereof.

The biomass may also be processed by homogenization, grinding, crushing, shredding, cutting up, carving, breaking, lyophilizing, digesting, fermenting, incubating, dessicating, and microbiologically, thermally, chemically, biochemically and/or biologically treating, and combination thereof, before solubilisation.

In accordance with the present invention there is provided a biomass under the form of a powder, an homogenate, a grinded, crushed, cutted up, carved, or broken biomass, a piece, and/or a part of biomass.

Another aim of the present invention is to provide microorganisms selected from the group consisting of bacteria, mould, yeast, Azotobacter, Peudomonas, Nocardia, Coliform, Alcaligenes, Bacillus, Lactobacillus, Burkholderia, Rhodococcum, Methylobacterium, and genetically modified form thereof.

More specifically, microorganisms may be Azotobacter chroococcum, Azotobacter vinelandii, Escherichia coli, Pseudomonas cepacia, Alcaligenes lipolytica, Pseudomonas oleovorans and Azotobacter salinestris.

This summary of the invention does not necessarily describe all variations of the invention, but that the invention may also reside in a sub-combination of these features described herein.

### BRIEF DESCRIPTION OF THE DRAWINGS

FIG. 1 illustrates the evolution of glucose concentration (g/l), cell dry weight (g/l) and PHA accumulation when conditions of example 1 are applied

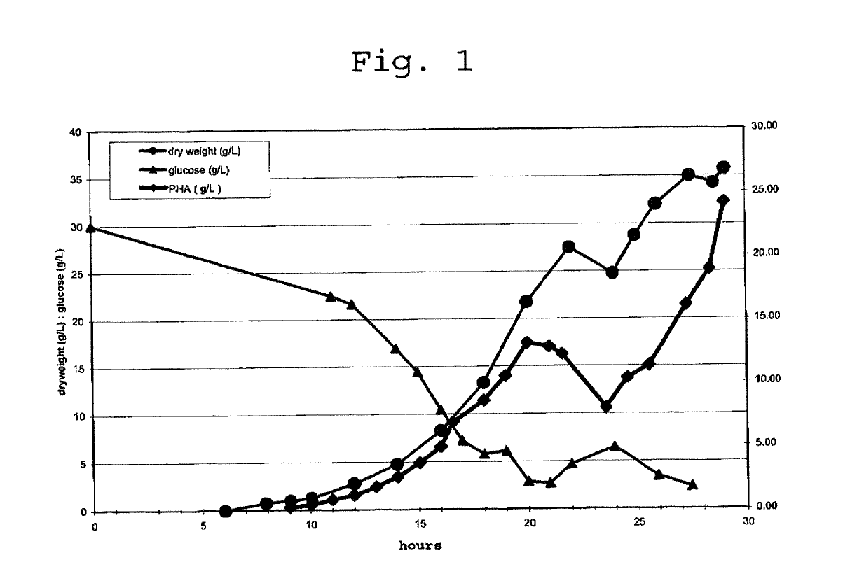

### DETAILED DESCRIPTION OF THE INVENTION

The following description is of a preferred embodiment by way of example only and without limitation to the combination of features necessary for carrying the invention into effect.

In accordance with the present invention, there is provided a process comprising fermentation conditions in which at least one PEA producing microorganism at high yields and/or output rates from starch or hydrolysable derivatives thereof as carbon source. Among derivatives that can be included, limiting the invention: chemically, biochemically, biologically and/or enzymatically modified starch and/or byproducts of starch.

One embodiment of the invention is to provide a process for producing PHAs, which comprises culturing at least one strain of PHA producing bacteria. The strains of PHA producing bacteria can be selected from the group of species consisting of Azotobacter, Pseudomonas, Nocardia, Alcaligenes, Bacillus, Lactobacillus, Methylobacterium, Rhodoccus, Burkholderia, Escherichia coli, and recombinant forms thereof. Other PHA producing microorganisms that can be considered, but without any limitation, in the present invention are yeasts, fungi and moulds.

A preferred embodiment of the invention is the use of bacteria Azotobacter salinestris, Azotobacter vinelandii, recombinant Escherichia coli, Pseudomonas cepacia, Pseudomonas oleovorans, Methylobacterium extorquens, Azotobacter chroococcum, and/or Alcaligenes eutrophus, or a mixture thereof, to perform the fermentation step in production of PHAs from starch.

The process of the present invention is applicable to recover PHA polymers produced by microorganisms either naturally or through genetic engineering, or PHAs that are synthetically produced. PHA is a polymer having the following general structure:

H—[O—CHR—(CH₂)p—CO]ₙ—OH

wherein R is preferably an H, alkyl, or alkenyl; p is 0, 1, 2, 3, 4, or 5; and n is an integer.

In another embodiment of the invention PHA may consist entirely of a single monomeric repeating unit, in which case it is referred to as a homopolymer. For example, polyhydroxybutyrate (PHB) homopolymer has repeating monomeric units where R is a methyl group and p=1. Copolymers, in contrast, contain two different types of monomeric units. PHBHV, for example, is a copolymer containing both polyhydroxybutyrate and hydroxyvalerate where R is an ethyl group, and p=1) units in variable ratios and incorporation order. Another copolymer of interest contains 3-hydroxybutyrate and 4-hydroxybutyrate units (P3HB4HB). When three different types of repeating units are present the polymer is referred to as a terpolymer.

Alternatively, biological synthesis of the biodegradable PHAs useful in the present invention may be carried out by fermentation with the proper organism (natural or genetically engineered) with the proper carbon source (single or multicomponent).

The PHA compositions produced according to one embodiment of the present invention can be recovered from the PHA-producing microorganism by conventional methods. Typically, a solvent-based approach is utilized, wherein the cells are harvested, dried, and the PHA is extracted with a solvent capable of dissolving PHA from other bacterial components. However, methods suitable for the recovery of PHAs from microbial and other biomass sources are also expected to be suitable for the recovery of analogs or modified forms of PHA made in accordance with the present invention.

In another embodiment of the present invention, there is provided a method of using the PHA of the present invention to produce a polymer or copolymer, wherein the PHA may be reacted with a coupling agent. The polymer or copolymer to produced could be, for example, a block, a random or graft polymer or copolymer thereof. Also provided are the polymer and copolymer compositions produced therefrom. Suitable coupling agents may include, for example, alkyl or aryl diisocyanate or triisocyanate, phosgene, alkyl or diaryl carbonate, a monomeric organic diacid, a monomeric organic diacid chloride, a monomeric organic diacid anhydride or a monomeric organic tetraacid dianhydride. Alternatively, the coupling agent can be an oligomer with end-groups that are reactive with chemically modified PHA, such as carboxy-terminated oligomeric polyesters or an isocyanate-terminated oligomeric polyol or polyester. This approach can be used, for example, to produce polyesters, copolyesters, polyester-carbonates, and polyester urethanes.

The most preferred PHA polymers for use in this invention are poly(hydroxybutyrate-co-hydroxyvalerate) polymers (PHBHPV), poly(3-hydroxybutyrate-co-4-hydroxybutyrate) copolymers (P3HS4HB), and hydroxyterminated polymers and copolymers of polyhydroxybutyrate (PHB-OH) and polyhydroxyalkanoate (PHA-OH).

According to a further embodiment of the present invention, there is provided a method of using the analogs and/or modified PHA of the present invention to produce a polymer of copolymer, wherein the PHA is reacted with a coupling agent and with a different modified moiety. The polymer so produced could be, for example, a block or random block polymer or copolymer. Also provided are the polymer and copolymer compositions produced therefrom. Suitable coupling agents may include, for example, alkyl or aryl diisocyanate or triisocyanate, phosgene, alkyl or diaryl carbonate, a monomeric organic diacid, a monomeric organic diacid chloride, a monomeric organic diacid anhydride or a monomeric organic tetraacid dianhydride. Alternatively, the coupling agent can be an oligomer with end-groups that are reactive with modified PHA, such as carboxy-terminated oligomeric polyester or polyamide, or a isocyanate-terminated oligomeric polyol, polyester or polyamide. A chemically modified moiety for use in this embodiment can include polyester diols such as polycaprolactone diol, polybutylene succinate diol, polybutylene succinate co-butylene adipate diol, polyethylene succinate diol, and similar aliphatic polymeric and copolymeric diols. Alternatively, the chemically modified moiety can be a polyesther diol such as a polyethylene oxide-diol, polypropylene oxide-diol, or polyethylene oxide-propylene oxide diol. This approach can be used, for example, to produce polyesters, copolyesters, polyester carbonates, polyester urethanes, polyester ethers, polyester amides, copolyester ethers, polyester ether carbonates, and polyester ether urethanes.

In a further embodiment of the present invention, there is provided a method of using the PHA or analogs thereof to produce a block polymer or copolymer, comprising the steps of reacting the PHA with a reactive monomer. Also provided are the PHA-containing copolymer compositions produced therefrom. Where needed, catalysts and other reactants known in the art to facilitate the reaction are used. The reactive monomer used in this embodiment can include, for example, alkyl epoxides such as ethylene oxide and propylene oxide, lactones such as caprolactone, butyrolactone, propiolactone, valerolactone, lactams such as caprolactam, and formaldehyde. This approach can be used to produce polyesters, copolyesters, polyester ethers, polyester amides, and polyester acetals.

According to one embodiment of the invention, all strains of microorganisms are cultured in a medium that may contain the following mineral salts: 0.6-3.0 mM magnesium sulfate, 10-200 μM ferrous sulfate, 1.0-6.0 mM potassium phosphate monobasic or 2-5 mM potassium phosphate dibasic, 0.7-32 μM sodium molybdate, 10-25 mM sodium chloride, and 0.4-1 mM calcium sulfate or calcium chloride.

In a particular embodiment, the salts medium contained may be 40-60 μM ferric citrate and 15-300 mM ammonium acetate. In one other case, the salts medium contained 1.5-2.5 mM sodium citrate and 30-300 mM ammonium nitrate.

According to another embodiment of the invention, 2-5% w/v of glucose from hydrolyzed starch solution having a DE (dextrose equivalent on a scale of 100) of 80 to 95 may he added to the medium.

On particular embodiment of the present invention is the biocompatibility of the PHA produced according to the process of the present invention. The commercial potential for PHAs of the invention opens up to important industries such as cosmeceutical, pharmaceutical and biomedical, and is derived primarily from a most advantageous property that distinguish PHA polymers from most petrochemical-derived polymers, namely biocompatibility. Biocompatibility may be defined as the quality of not having toxicological effects on biological systems and/or the ability of a material to perform a specific application with this same quality. This quality allows for numerous applications such as drug delivery, orthopedic implant, tissue engineering and cardiovascular uses.

### Material and Methods

Microorganism and Culture Media

The strain used for the production of PHA is Azotobacter salinestris (ATCC 49674). Azotobacter salinestris is a gram-negative bacteria related to Azotobacter chroococcum and is cultured in a medium as described above.

The fermentor inoculum consists in a pre-grown (18-24) culture with a corresponding cell dry weight of 1-5 g/l. Samples of quickly halted log growth phase are mixed with an equal volume of glycerol 30% (v/v) and stored in vials (1-2 ml) at −80° C. to constitute a working cells bank.

Potato Starch Hydrolysis

Potato tubers or peels are first washed and shredded. Water is then added to form 500-2000 g/l potato slurry depending on final glucose concentration desired. The resulting mixture may then be subjected to starch hydrolysis, which is a two steps process. In the first one, called liquefaction, the starch slurry is heat treated (65-95° C. at 350 rpm for 30 min-1 h), before being hydrolyzed to a maltodextrines solution with a heat-stable α-amylase enzyme preparation (Termamyl®120L, Novo Nordisk) in presence of calcium ions. This step is carried out directly in a steamed tank reactor vessel equipped with temperature, stirrer speed and pH adjustments all of which set at the following operating parameters. 90-100° C.; 200-350 rpm; pH=6.0-6.5 for a period of up to 60-120 min. The pH may be adjusted with calcium hydroxide to provide the necessary calcium ions. The second step, called saccharification, allows for further hydrolysis of the dextrines into glucose. It is performed with a 1,4-alpha-D-glucohydrolase (AMG 300, Novo Nordisk) after setting the operating parameters as: 55-60° C.; 200-250 rpm; pH=4.2-4.8 for a period of 24-60 h. The degree of enzymatic hydrolysis may be determined with the use of a rapid analysis system for the glucose concentration (Biolyzer by Kodak, New Haven, Conn.).

Fed-Batch Culture

Fermentation is performed in a conventional controlled stirred tank reactor (STR) at 25-30° C. and pH=7.0. The fermentation media is the same as the one described above for the cultivation of the microorganism. The fermentor is seeded with a 2-10% (v/v) fresh inoculum in active growth phase. The agitation and airflow rate are varied during course of fermentation to maintain the dissolved oxygen level (DO) above 3-5% saturation and preferably around 5-10% saturation. Following a log phase of 4-10 h, it is necessary to maintain the glucose level by feeding with a hydrolyzed starch stock solution at a concentration of 20-80% w/v glucose at a variable feed rate in the range of 5-10 ml/l/h. Fish peptone, modified meat peptone, or yeast extract may be also supplied to the growth medium to enhance PHB synthesis. Peptones are thought to act as a PHA yield promotion factor at concentration of 0.05 to 0.2% w/v. For best results, the peptone solution should be added at a rate proportional to the glucose supplement. It is also required to maintain a continuous supply of broth nutrient by feeding a concentrate of the fermentation medium throughout the growth phase. A typical feedstock may consist of a 4-20 times the initial broth concentration and should be supplied at a rate proportional to glucose feed solution. At the end of fermentation, cells are separated from the spent medium by centrifugation or filtration.

Polymer Extraction Method

PHA isolation consists in a step procedure in which cells are sequently separated, washed and then submitted to polymer extraction as described. Cells are washed once or twice in distilled water and membranes are broken by using hot mixture of NaOH and NH₄OH or NaOH, NH₄OH and SS or NaOH, NH₄OH and Triton™, or mechanically by glass beads or other shear forces or by heat treatment. PHA is then isolated using different approaches such as solvent extraction using chloroform or methylene dichloride or by digesting NPCM (non polymer cell material) using enzyme cocktail of protease, lipase and nuclease. PHA is finally recovered by centrifugation, differential centrifugation or filtration, and dried avoiding direct light exposure. Physical determination such as average molecular weight and polydispersivity index may be carried out using standard procedures known in the art.

### EXAMPLE I

### Growth of A. salinestris and Production of PRA Following a Fedbatch Fermentation Strategy

An inoculum of A. salinestris (strain ATCC 49674) was grown aerobically in a 2 liters Fernback™ flask containing 500 ml of previously described culture medium. The flask was incubated at 30° C. for 24 h with rotating agitation set at 250 rpm.

The resulting inoculum was then added to a 14 liters bioreactor (CHEMAP) containing 8 liters of the previously described fermentation medium. The fermentation was carried out at 30° C. in a fed-batch mode at the following conditions: 1) the pH was maintained at 7 using concentrated solution of sodium hydroxyde or sulfuric acid; 2) the aeration rate and the agitation speed were adjusted manually during course of fermentation to maintain the level of oxygen above 5% and below 30% saturation. The maximum agitation speed reached was 610 rpm; 3) foam formation was controlled with addition of MAZU™ (PPG Industries); 4) glucose was fed throughout growth phase from 20-80% w/v stock solution as obtained by starch hydrolysis, at a rate of approximately 5-10 ml/l/h; 5) spent nutrients were provided throughout growth phase by feeding a 4-20 times concentrated fermentation medium. Feed rate was approximately 5-10 m/l/h. The fermentation was stopped after 30 hours.

The PHA was recovered using modified method of Berger (Berger et al. (1989) Biotechnology Techniques, 3:227-232). Cells were centrifuged 15 minutes at 3000×g and then washed twice in distilled water. 50 ml of methanol were added to an equivalent of 5 g (dry weight) of cells and vigorously mixed. The mixture was incubated 48 h at 40° C. and the cells were harvested by centrifugation at 3000×g for 15 minutes. The supernatant was discarded and 100 ml of chloroform was added to the pellet. The mixture was gently agitated and incubated at 40° C. for 24 h. 100 ml of distilled water was added to the chloroform mixture, carefully agitated and centrifuged at 3000×g for 15 minutes. The lower phase was recuperated and the soluble polymer precipitated with the addition of cold ethanol 95% under continuous agitation. The precipitated PHA obtained was recovered by filtration and dried at room temperature avoiding light exposure.

At the end of the fermentation, the cell biomass concentration was 30-40 g/l (dry weight), containing approximately 15-20 g/l of PHB/HV (92% HB and 8% HV) with a molecular weight of 1 million and a polydispersity index of 1.2.

### EXAMPLE II

### Production of Copolymer PHB/HV Following a Co-Substrate Fedbatch Fermentation Strategy

A inoculum of A. salinestris (ATCC 49674) was grown aerobically in a 2 liters flask containing 500 ml of previously described culture medium supplemented with 30 mM sodium valerate. The culture was incubated at 30° C. for 24-30 h rotating agitation set at 250 rpm.

The fermentation parameters were similar to those described in Example 1 for the aeration rate, pH and dissolved oxygen level. Sodium valerate as well as glucose were added during course of fermentation from a concentrate of 500 mM sodium valerate and 50% glucose in order to obtain a random copolymer of 3HB-3HV or a block copolymer. Depending on the feed strategy, copolymers were composed of 65 to 90% of HB and 10 to 35% of HV, with a MW of 1 million and P.I. of 1.2.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A process for production of polyhydroxyalkanoate (PHA) from starch and/or derivatives thereof which comprises the step of incubating at least one strain of PHA-producing microorganism for a sufficient period of time and conditions to produce said PHA in a culture medium comprising starch and/or a derivative thereof.

2. The process according to claim 1, wherein said starch is isolated from a starch-containing biomass, said biomass being processed to render said starch sufficiently available to be chemically, biochemically, biologically or enzymatically treated.

3. The process according to claim 1 or 2, which further comprises the step of isolating said PA from said microorganism and/or said medium.

4. The process according to claim 2, wherein said biomass is selected from the group consisting of a plant, wastewater, wash water, a potato, and a by-products or a derivative thereof.

5. The process according to claim 1, wherein said starch is selected from the group consisting of a synthetic, a crude, a chemically, a biochemically, a biologically, and a enzymatically treated starch.

6. The process according to claim 2, wherein said starch is hydrolyzed starch.

7. The process according to claim 1, wherein said polyhydroxyalkanoate is selected from the group consisting of a polymer of a hydroxyalkanoic acid, a hydroxybutyric acid, a hydroxyvaleric acids and a copolymer thereof.

8. The process according to claim 7, wherein said copolymer is a poly(hydroxybutyrate-co-hydroxyvalerate) (PHBHV), a poly(3-hydroxybutyrate-co-4-hydroxybutyrate) (P3HB4HB), a polymer and/or a copolymer of hydroxyterminated polyhydroxybutyrate (PHB-OH) a heteropolymer thereof, or a polymer having a chemical structure H—[O—CHR—(CH₂)ₚ—CO]ₙ—OH, wherein R is an H, alkyl, or alkenyl; p is 0, 1, 2, 3, 4, or 5; and n is an integer.

9. The process according to claim 2, wherein said process to render said starch sufficiently available from said processed biomass is selected from the group consisting of homogenizing said starch, grinding said starch, crushing said starch, shredding said starch, cutting up said starch, carving said starch, breaking said starch, solubilizing said starch, lyophilizing said starch, digesting said starch, fermenting said starch, incubating said starch, dessicating said starch, microbiologically treating said starch, thermally treating said starch, chemically treating said starch, biochemically treating said starch, and biologically treating said starch, or a combination thereof.

10. The process according to claim 1, wherein said microorganism is selected from the group consisting of bacteria, mould, yeast, Azotobacter, Pseudomonas, Nocardia, Coliform, Alcaligenes, Bacillus, Lactobacillus, Burkholderia, Rhodococcum, and Methyylobacterium, or a genetically modified form thereof.

11. The process according to claim 1, wherein said microorganism is Azotobacter chroococcum, Azotobacter vinelandii, recombinant Escherichia coli, Pseudomonas cepacia, Pseudomonas oleovorans, or Alcaligenes lipolytica.

12. The process according to claim 1, wherein said microorganism is Azotobacter salinestris.

13. A polyhydroxyalkanoate (PHA) produced by incubation of at least one strain of PRA-producing microorganism in a culture medium comprising starch and/or a derivative thereof.

14. The PHA according to claim 13, wherein said biomass is selected from the group consisting of plant, wastewater, wash water, potato, and by-products or a derivative thereof.

15. The PHA according to claim 13, wherein said starch is selected from the group consisting of a synthetic, a crude, a chemically, a biochemically, a biologically, and an enzymatically treated starch.

16. The PHA according to claim 13, wherein said starch is hydrolyzed starch.

17. The PHA according to claim 13, wherein said PHA is selected from the group consisting of a polymer of hydroxyalkanoic acid, hydroxybutyric acid, hydroxyvaleric acid, and a copolymer thereof.

18. The PHA according to claim 17, wherein said copolymer is a poly(hydroxybutyrate-co-hydroxyvalerate) (PHBHV), a poly(3-hydroxybutyrate-co-4-hydroxybutyrate) (P3HB4HB), a polymer and/or a copolymer of hydroxyterminated polyhydroxybutyrate (PHB-OH), a heteropolymer thereof, or a polymer having a chemical structure H—[O—CHR—(CH₂)ₚ—CO]ₙ—OH, wherein R is an H alkyl, or alkenyl; p is 0, 1, 2, 3, 4, or 5; and n is an integer.

19. The PHA according to claim 13, wherein said microorganism is selected from the group consisting of a bacteria, a mould, and a yeast.

20. The PHA according to claim 13, wherein said microorganism is selected from the group consisting of Azotobacter, Pseudomonas, Nocardia, Coliform, Alcaligenes, Bacillus, Lactobacillus, Burkholderia, Rhodococcum, and Methylobacterium, or a genetically modified form thereof.

21. The PHA according to claim 13, wherein said microorganism is Azotobacter chroococcum, Azotobacter vinelandii, recombinant Escherichia coli, Pseudomonas cepacia, Pseudomonas oleovorans, or Alcaligenes lipolytica.

22. The PHA according to claim 13, wherein said microorganism is Azotobacter salinestris.
